(19) United States
(12) Patent Application Publication
(10) Pub. No.: US 2006/0194840 A1
(43) Pub. Date: Aug. 31, 2006

(54) Method for treating snoring and sleep apnea with leukotriene antagonists

(76) Inventor: David Gozal, Louisville, KY (US)

Correspondence Address: STITES & HARBISON PLLC, 424 CHURCH STREET, SUITE 1800, NASHVILLE, TN, 37219-2376, US

(21) Appl. No.: 11/385,583

(22) Filed: Mar. 20, 2006

Related U.S. Application Data
(63) Continuation-in-part of application No. PCT/US04/30877, filed on Sep. 20, 2004.
(60) Provisional application No. 60/504,149, filed on Sep. 19, 2003.

(51) Int. Cl. A61K 31/47

(52) U.S. Cl. 514/311

(57) ABSTRACT

A method of treating snoring and/or sleep apnea comprising administering to a patient in need of such treatment a therapeutically effective amount of a leukotriene receptor antagonist.

### PRIORITY INFORMATION

This application is a continuation application under 35 U.S.C. § 120 of PCT International Application Number PCT/US04/30877, filed Sep. 20, 2004, which claims priority to U.S. Application No. 60/504,149, filed Sep. 19, 2003, now abandoned. The contents of both application are incorporated herein by reference.

### TECHNICAL FIELD OF THE INVENTION

The present invention relates generally to leukotriene antagonists and, more particularly to methods for use thereof in the treatment of snoring and sleep apnea.

### BACKGROUND ART AND BACKGROUND OF THE INVENTION

The leukotrienes are a group of locally acting hormones, produced in living systems from arachidonic acid. Leukotrienes have been associated with inflammatory cells and have been recognized as spasmogens for bronchial smooth muscle, thus, they have been implicated as a trigger for asthmatic episodes. Details of the biosynthesis and metabolism of the leukotrienes, as well as the actions of the leukotrienes in living systems and their contribution to various diseases states, may be found in Leukotrienes and Lipoxygenases, ed. J. Rokach, Elsevier, Amsterdam (1989), which is incorporated herein by reference.

Certain leukotriene antagonists have been used as anti-asthmatic, anti-allergic, anti-inflammatory, and cytoprotective agents. Examples of leukotriene antagonists may be found in U.S. Pat. No. 5,565,473 to Belley et al, which is incorporated herein by reference in its entirety, as part of this description.

Although certain leukotriene antagonists have been used for treatment of ailments, such as asthma, they have not heretofore been used in the treatment of sleep apnea or snoring. As will be discussed in the detailed description of the invention, the applicant has found leukotriene antagonists to be beneficial in the treatment of sleep apnea and snoring.

Leukotrienes are naturally-occurring molecules that function as inter-cellular messengers in mammals. There are several subtypes, referred to by designations such as LTA₄, LTB₄, LTC₄, LTD₄, and LTE₄.

All of these subtypes are formed from arachidonic acid, a molecule containing 20 carbon atoms, which has four internal double bonds near the center of the chain and a carboxylic acid group at one end. Arachidoric acid is continuously synthesized at cell membranes, by cleavage of certain types of phospholipids. This cleavage reaction is catalyzed by phospholipase enzymes. The free arachidonic acid is then converted into any of four different types of compounds, which are leukotrienes, prostaglandins, prostacyclins, and thromboxanes. All four of these types of compounds are called “eicosanoids”.

Prostaglandins, prostacyclins, and thromboxanes all contain cyclic structures, and are created when “cyclooxygenase” enzymes (often abbreviated as COX enzymes) generate these cyclic structures from the carbon chain in arachidonic acid.

By contrast, leukotrienes are created by the action of different types of enzymes. Initially, one of the four double bonds in arachidonic acid is converted into an epoxide structure; the three double bonds that remain give leukotrienes the “tri-ene” classification. The epoxide structure in LTA₄ is relatively reactive and unstable, so LTA₄ serves mainly as a precursor during synthesis of the other leukotrienes. LTB₄ is generated ashen the epoxide form is been hydrolyzed into a di-hydroxy compound, while LTC₄, LTD₄ and LTE₄ are all modified by the addition of cysteine, an amino acid that contains a relatively reactive sulfhydryl group (—SH) at the end of a spacer chain.

All of the eicosanoid compounds tend to aggravate inflammatory, pain, and fever responses, and they have been the targets of extensive research on anti-inflammatory and analgesic drugs. For example, anti-inflammatory steroids such as cortisone function by suppressing the phospholipase enzymes that generate arachidonic acid from membrane phospholipids. Pain-killers such as aspirin and ibuprofen act by blocking (to some extent) the cyclooxygenase enzymes that control the conversion of arachidonic acid to prostaglandins, prostacyclins, and thromboxanes.

Leukotrienes have been recognized as inflammatory agents since the early 1980's. In the 1990's, various drug; known as “leukotriene antagonists” were identified, which can suppress and inhibit the activity of leukotrienes in the body.

The term “leukotriene antagonist” (LT) is used herein in the conventional medical sense, to refer to a drug that suppresses, blocks, or otherwise reduces or opposes the concentration, activity, or effects of one or more subtypes of naturally occurring leukotrienes. In laymen's terms, LT antagonists can be referred to as LT blockers.

LT antagonist drugs can wore by any of at least three distinct mechanisms: (i) by inhibiting the enzymes that convert arachidonic acid into leukotrienes; (ii) by competitively occupying leukotriene receptors on the surfaces of cells, thereby making those receptors unavailable to react with leukotrienes, without triggering (“agonizing”) the cellular reactions that are triggered by leukotrienes; or (iii) by binding to leukotriene molecules in blood or other body fluids, thereby entangling or altering the leukotriene molecules and rendering them unable to trigger leukotriene receptors.

Two LT antagonist drugs have become successful and widely used treatments for asthma, since they can help suppress the bronchial and alveolar constrictions that cause or aggravate asthma attacks. Those two drugs are: (i) zafirlukast, which is sold under the tradename “Accolate” by Zeneca Pharmaceuticals (Wilmington, Del.), and (ii) montelukast, sold under the tradenames “Singulair” by Merck and Company (West Point, Pa.). Various other LT antagonist drugs are also known, such as pranlukast, BAYx7195, LY293111, ICI 204,219, and ONO-1078. All of these LT antagonist drugs listed above are believed to help control and suppress asthma attacks primarily by competitive binding to (and blocking of) one or more types of leukotriene receptors on bronchial cells and various types of blood cells.

In addition, various drugs are known which can inhibit the synthesis of LT molecules, by inhibiting one or more of the lipoxygenase enzymes that synthesize LT molecules. Such drugs include BAYx1005, MK-886, MK-0591, ZD2138, and zileuton (also known as A-64077).

Accolate and Singulair are both sold in pill form, and can be taken every day for long periods of time. Rather than creating tolerance or dependence problems, these drugs appear to help suppress and reduce ongoing asthma problems, when taken chronically, by helping suppress the hypersensitive immune or allergic responses that often grow cumulatively worse in people who suffer from unwanted and excessive activity of the allergic or other immune systems.

As noted above, leukotriene antagonists have not previously been used to treat or prevent snoring or obstructive sleep apnea. Instead, there is a need for a treatment that can be used on a chronic and long-term basis, to prevent those and the other related indications disclosed herein.

Accordingly, one object of the subject invention is to disclose and provide a method for long-term and chronic yet safe administration of a drug that can prevent snoring and obstructive sleep apnea.

Obstructive sleep apnea is a breathing disorder caused by a blockage of the airway and is characterized by fragmented sleep patterns caused by brief arousals for the purpose of recommencing breathing. Obstruction of the airway is caused in a variety of manners, for example, the tonsils or adenoids may become large enough, relative to the airway size, to cause or contribute to a blockage of air flow through the airway.

Sleep apnea is a common disorder affecting more than twelve million American adults and children, according to the National Institutes of Health. Sleep apnea sufferers, because of their fragmented sleep patterns, experience many problems which correlate to their sleep deprivation, for example, day-time exhaustion, depression, irritability, memory difficulties. Those with sleep apnea can also experience problems with heavy snoring. Additionally, symptoms may be even more severe, for example, the risk for a heart attack and stroke are increased for those suffering from sleep apnea.

Treatment options include continuous positive airway pressure (CPAP), which involves the sleep apnea sufferers wearing masks over their noses and having air forced through their nasal passages. In addition to the obvious drawbacks of this treatment, side effects include nasal irritation and drying, abdominal bloating, and headaches.

Other treatment options involve surgery. For example, adenotonsillectomy, removal of the adenoids and the tonsils, is a procedure commonly preformed as a treatment for sleep apnea. Because of the severity of surgical treatment options, not all sleep apnea sufferers are considered appropriate candidates for surgery. For example, there is not a consensus on whether certain patients having an apnea-hypopnea index of less than five (5) are appropriate candidates for surgery. Apnea-hypopnea index (AHI) is a measure of the number of apneic and hypopneic episodes combined per hour of sleep. An apneic episode is generally considered a cessation of breathing while a hypopneic episode is generally considered an abnormal decrease in the depth and rate of breathing. Accordingly, for patients having fewer than five (5) apneic and hypopneic episodes combined per hour of sleep, surgery is often considered inappropriate.

Nasal steroids have also been used to treat sleep apnea and snoring, but are only effective while the treatment is continued. Unfortunately, continual use is not recommended. Indeed, over time, the steroid treatment may result in habituation and rebound growth of adenoid tissue may occur when the steroid treatment is discontinued, compounding the problem.

It is therefore the primary object of the present invention to provide a method for treating snoring and sleep apnea with leukotriene antagonists, which do not have the drawbacks of known treatment methods. The efficacy of leukotriene antagonists for the treatment of snoring and sleep apnea will be described below.

This and other objects and advantages of the present invention will become apparent upon a reading of the following description when taken in conjunction with the accompanying drawings.

### DETAILED DESCRIPTION OF THE INVENTION

The present invention is a method for treating and/or preventing snoring and sleep apnea with leukotriene antagoinists. As mentioned, enlarged tonsils or adenoids can cause or contribute to a blockage of air flow through a patient's airway causing the patient to suffer from sleep apnea, snoring, or both. As also mentioned, leukotrienes have been associated with inflammatory cells and, indeed, are produced by certain inflammatory cells. For cells having leukotriene receptors, the binding of leukotriene to the receptors can cause inflammation and enlarging of the tissue being comprised of those cells.

By definition, leukotriene antagonists have the ability to compete with leukotrienes for receptor binding sites, and, if present in an effective concentrations, can prevent or reverse the symptoms induced by the leukotrienes. Without being bound by theory, the inflammation and enlarging of the tonsils and adenoids in certain sleep apnea sufferers may be due to the presence of leukotriene receptors in the adenotonsillar tissue of the sufferers and the binding of leukotriene thereto.

Accordingly, one method of the present invention proposes to administer leukotriene antagonists to prevent or reverse any leukotriene-induction inflammation in the tonsils and adenoids of a patient having sleep apnea, thereby eliminating or relieving the blockage of air flow through the patient's airway resulting from enlarged tonsils or adenoids.

The method of the present invention may be practiced, for example, by administering an appropriate pharmaceutical composition of a leukotriene antagonist, such as those described in U.S. Pat. No. 5,565,473, in an effective amount, which may be the doses described in the '473 patent.

The compositions of the present invention thus include compounds of the following formula: 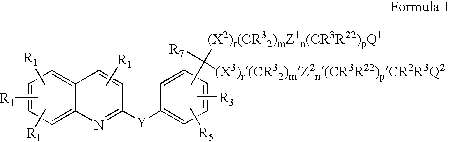
wherein:

R₁ is H, halogen, —CF₃, —CN, —NO₂, or N₃;

R₂ is lower alkyl, lower alkenyl, lower alkynyl, —CF₃, —CH₂F, —CH₂F₂, CH₂CF₃, substituted or unsubstituted phenyl, substituted or unsubstituted benzyl, substituted or unsubstituted 2-phenethyl, or two R² groups joined to the same carbon may form a ring of up to 8 members containing 0-2 heteroatoms chosen from O, S, and N;

R³ is H or R²;

CR³R²² may be the radical of a standard amino acid;

R⁴ is halogen, —NO₂, —CN, —OR₃, —SR³, NR³R³, NR³C(O)R⁷ or R³;

R⁵ is H, halogen, —NO₂, —N₃, —CN, —SR², —NR³R³, —OR³, lower alkyl, or —C(O)R³;

R⁶ is (CH₂)ₛ—C(R⁷R⁷)—CH₂)ₛ—R⁸ or —CH₂C(O)NR¹²R¹²;

R⁷ is H or C₁₋₄ alkyl;

R⁸ is
A) a monocyclic or bicyclic heterocyclic radical containing from 3 to 12 nuclear carbon atoms and 1 or 2 nuclear heteroatoms selected from N, S or O and with each ring in the heterocyclic radical being formed of 5 or 6 atoms, or
B) the radical W—R⁹;

R⁹ contains up to 20 carbon atoms and is (1) an alkyl group or (2) an alkylcarbonyl group of an organic acyclic or monocyclic carboxylic acid containing not more than 1 heteroatom in the ring;

R¹⁰ is —SR¹¹, —OR¹², or —NR¹²R¹²;

R¹¹ is lower alkyl, —C(O)R¹⁴, unsubstituted phenyl, or unsubstituted benzyl;

R¹² is H, R¹¹ or two R¹² groups joined to the same N may form a ring of 5 or 6 members containing 1-2 heteroatoms chosen from O, S, and N;

R¹³ is lower alkyl, lower alkenyl, lower alkynyl, —CF₃ or substituted or unsubstituted phenyl, benzyl, or 2-phenethyl;

R¹⁴ is H or R¹³;

R¹⁶ is H, C₁₋₄ alkyl, or OH;

R¹⁷ is lower alkyl, lower alkenyl, lower alkynyl, or substituted or unsubstituted phenyl, benzyl, or 2-phenethyl;

R¹⁸ is lower alkyl, lower alkenyl, lower alkynyl, —CF₃ or substituted or unsubstituted phenyl, benzyl, or 2-phenethyl;

R¹⁹ is lower alkyl, lower alkenyl, lower alkynyl, —CF₃ or substituted or unsubstituted phenyl, benzyl, or 2-phenethyl;

R²⁰ is H, C₁₋₄ alkyl, substituted or unsubstituted phenyl, benzyl, phenethyl, or pyridinyl or two R²⁰ groups joined to the same N may form a saturated ring of 5 or 6 members containing 1-2 heteroatoms chosen from O, S, and N;

R²¹ is H or R¹⁷;

R²² is R⁴, CHR⁷OR³, or CHR⁷SR²;

m and m′ are independently 0-8;

n and m′ are independently 0 or 1;

p and p′ are independently 0-8;

m+n+p is 1-10 when r is 1 and X² is O, S, S(O), or S(O)₂;

m+n+p is 0-10 when r is 1 and X² is CR³R¹⁶;

m+n+p is 0-10 when r is O;

m′+m′+p′ is 0-10;

r and r′ are independently 0 or 1;

s is 0-3;

Q¹ is —C(O)OR³, 1H(or 2H)-tetrazol-5-yl, —C(O)OR⁶, —C(O)NHS(O)₂R¹³, —CN, —C(O)NR¹²R¹², —NR²¹S(O)₂R¹², —CN, —NR¹²C(O)NR¹²R²¹, —NR²¹C(O)R¹⁸, —OC(O)NR¹²R¹²—, C(O)R¹⁹, —S(O)R¹⁸, —S(O)₂R¹⁸, —S(O)₂NR¹²R¹², —NO₂, —NR²¹C(O)OR¹⁷, —C(R¹²R¹²)═NR¹², —C(R¹³)═NOH; or if Q¹—C(O)OH and R²² is —OH, —SH, —CHR⁷ OH or —NHR³, then Q¹ and R²² and the carbons through which they are attached may form a heterocyclic ring by loss of water;

Q² is OH or NR²⁰R²⁰;

W is O, S, or NR³;

X² and X³ are independently O, S, S(O), S(O)₂, or CR³R¹⁶;

Y is —CR³═CR³— or —C═C—;

Z¹ and Z² are independently —HET(—R³—R⁵)—;

HET is the diradical of a benzene, a pyridine, a furan, or a thiophene;

and the pharmaceutically acceptable salts thereof.

### DEFINITIONS

The following abbreviations have the indicated meanings:

Et=ethyl

Me=methyl

Bz=benzyl

Ph=phenyl

t-Bu=tert-butyl

i-Pr=isopropyl

n-Pr=normal propyl

c-Hex=cyclohexyl

c-Pr=cyclopropyl

1,1-c-Bu=1,1-bis-cyclobutyl

1,1-c-Pr=1,1-bis-cyclopropyl (e.g., HOCH₂ (1,1-c-Pr)CH₂CO₂Me is methyl 1-(hydroxymethyl)cyclopropaneacetate)

c-=cyclo

Ac=acetyl

Tz=1H(or 2H)-tetrazol-5-yl

Th=2- or 3-thienyl

C₃H₅=allyl

c-Pen=cyclopentyl

c-Bu=cyclobutyl

phe=benzenediyl

pye=pyridinediyl

fur=furandiyl

thio=thiophenediyl

DEAD=diethyl azocarboxylate

DHP=dihydropyran

DIAD=diisopropyl azodicarboxylate

r.t.=room temperature

Alkyl, alkenyl, and alkynyl are intended to include linear, branched, and cyclic structures and combinations thereof.

“Alkyl” includes “lower alkyl” and extends to cover carbon fragments having up to 20 carbon atoms. Examples of alkyl groups include octyl, nonyl, norbornyl, undecyl, dodecyl, tridecyl, tetradecyl, pentadecyl, eicosyl, 3,7-diethyl-2,2-dimethyl-4-propylnonyl, 2-(cyclododecyl)ethyl, adamantyl, and the like.

“Lower alkyl” means alkyl groups of from 1 to 7 carbon atoms. Examples of lower alkyl groups include methyl, ethyl, propyl, isopropyl, butyl, sec- and tert-butyl, pentyl, hexyl, heptyl, cyclopropyl, cyclobutyl, cyclopentyl, cyclohexyl, cycloheptyl, 2-methylcyclopropyl, cyclopropylmethyl, and the like.

“Lower alkenyl” groups means alkenyl groups of 2 to 7 carbon atoms. Examples of lower alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, and the like.

“Lower alkynyl” means alkynyl groups of 2 to carbon atoms. Examples of lower alkynyl groups include ethynyl, propargyl, 3-methyl-1-pentynyl, 2-heptynyl, and the like.

“Alkylcarbonyl” means alkylcarbonyl groups of 1 to 20 carbon atom of a straight, branched or cyclic configuration. Examples of alkylcarbonyl groups are 2-methylbutanoyl, octadecanoyl, 11-cyclohexylundecanoyl and the like. Thus, the 11-cyclohexylundecanoyl group is c-Hex-(CH₂)₁₀—C(O)—.

Substituted phenyl, benzyl, 2-phenethyl and pyridinyl means structures with 1 or 2 substituents on the aromatic ring selected from lower alkyl, R¹⁰, NO₂, SCF₃, halogen, —C(O)R⁷, —C(O)R¹⁰, CN, CF₃, and CN₄H.

Halogen means F, Cl, Br and I.

The prodrug esters of Q¹ (i.e., when Q¹=—C(O)OR⁶) are intended to mean the esters such as are described by Saari et al., J. Med. Chem., 21, No. 8, 746-753 (1978), Sakamoto et al., Chem. Pharm. Bull., 32, No. 6, 2241-2248 (1984) and Bundgaard et al., J. Med. Chem., 30, No. 3, 451-454 (1987). Within the definition of R⁸, some representative monocyclic or bicyclic heterocyclic radicals are: 2,5-dioxo-1-pyrrolidinyl, (3-pyridinylcarbonyl)amino, 1,3-dihydro-1,3-dioxo-2H-isoindol-2-yl, 1,3-dihydro-2H-isoindol-2-yl, 2,4-imidazolinedion-1-yl, 2,6-piperidinedion-1-yl, 2-imidazolyl, 2-oxo-1,3-dioxolen-4-yl, piperidin-1-yl, morpholin-1-yl, and piperazin-1-yl.

When Q¹ and R²² and the carbons through which they are attached form a ring, the rings thus formed include lactones, lactams, and thiolactones.

It is intended that the definitions of any substituent (e.g., R¹, R², m, X, etc.) in a particular molecule be independent of its definitions elsewhere in the molecule. Thus, —NR³R³ represents —NHH, —NHCH₃, —NHC₆H₅, etc.

The heterocycles formed when two R³, R¹², or R²⁰ groups join through N include pyrrolidine, piperidine, morpholine, thiamorpholine, piperazine, and N-methylpiperazine.

“Standard amino acids”, the radical of which may be CR³R²², means the following amino acids: alanins, asparagine, aspattic acid, arginine, cysteine, glutamic acid, glutamine, glycine, histidine, isoleucine, leucine, lysine, methionine, phenylalanine, proline, serine, threonine, tryptophan, tyrosine, and valine. (See F. H. C. Crick, Symposium of the Society of Experimental Biology, 12, 140 (1958)).

Some of the compounds described herein contain one or more centers of asymmetry and may thus give rise to diastereoisomers and optical isomers. The present invention is meant to comprehend such possible diastereoisomers as well as their racemic and resolved, optically active forms. Optically active (R) and (S) isomers may be resolved using conventional techniques.

Some of the compounds described herein contain olefinic double bonds, and unless specified otherwise, are meant to include both E and Z geometric isomers.

In another aspect of the present invention, the method for treating snoring and sleep apnea comprises administering montelukast, [R-(E)]-1-[[[1-[3-[2-(7-Chloro-2-quinolinyl)ethenyl]phenyl]-3-[2-(1-hydroxy-1-methylethyl)-phenyl]propyl]thio]methyl]cyclopropaneacetic acid, a compound of the following structural formula: 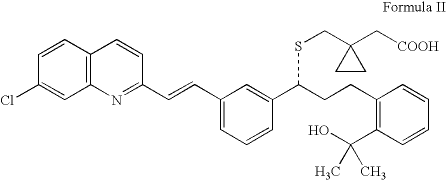
and stereoisomers, analogs, and pharmaceutical salts thereof.

In another aspect of the present invention, the methods disclosed herein comprise the use of the following compound: 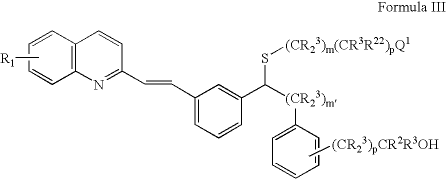
wherein:

R¹ is H, halogen, CF₃, or CN;

R²² is R³, —CH₂O₃, or —CH₂SR²;

Q¹ is —C(O)OH, 1H(or 2H)-tetrazol-5-yl, —C(O)NHS(O)₂R¹³, —C(O)NR¹²R¹², or —NHS(O)₂R¹³;

m′is 0, 1, 2 or 3;

p′ is 0 or 1;

m+p is 1-5;

the remaining definitions are as in Formula I;

and stereoisomers, analogs, and pharmaceutically acceptable salts thereof.

In another aspect of the present invention, the methods disclosed herein comprise the use of the following compound: 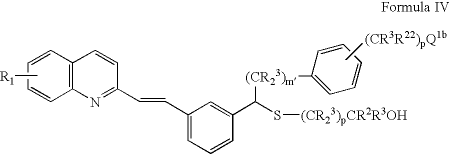
wherein:

R¹ is H, halogen, CF₃, or CN;

R²² is R³, —CH₂O₃, or —CH₂SR²;

Q¹ is —C(O)OH, 1H(or 2H)-tetrazol-5-yl, —C(O)NHS(O)₂R¹³, —C(O)NR¹²R¹², or —NHS(O)₂R¹³;

m′is 0, 1, 2 or 3;

p is 0 or 1

p′ is 1-4;

m+p is 0-4;

the remaining definitions are as in Formula I;

and the pharmaceutically acceptable salts thereof.

In another aspect of the present invention, the methods disclosed herein comprise the use at least one of the following LT antagonists of the present invention: monetlukast, zafirlukast, pranlukast, sodium 1-(((R)-3-(2-(6,7-difluoro-2-quinolinyl)ethenyl)phenyl)-3-(2-(2-hydroxy-2-propyl)phenyl)thio)methyl)cyclopropaneacetate, 1-(((1 (R)-3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid.

The following table illustrates compounds for use with the methods of the present invention of the present invention. These compounds are presented for exemplary purposes only and are not intended to be limiting of the present invention.

TABLE 1

| | |
|---|---|
| | I′ |
| 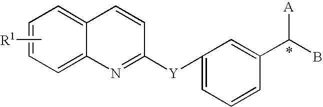 | |

| EX. | * | R¹ | Y | A | B |
|---|---|---|---|---|---|
| 1 | RS | 7-Cl | C≡C | SCH₂CHMeCO₂H | (CH₂)₂(1,2-phe)CMe₂OH |
| 2 | RS | 7-Cl | CH═CH | S(CH₂)₂CO₂H | (CH₂)₂(1,2-phe)C((CH₂)₄)OH |
| 3 | RS | 7-Cl | CH═CH | S(CH₂)₂CO₂H | (CH₂)₂(4-Cl-1,2-phe)CMe₂OH |
| 4 | RS | 7-Cl | CH═CH | SCH₂CHMeCO₂H | (1,3-phe)CMe₂OH |
| 5 | RS | 7-Cl | CH═CH | S(CH₂)₂CO₂H | (CH₂)₂(1,2-phe)CMe₂OH |
| 6 | RS | 7-Cl | CH═CH | SCH₂CHMeCO₂H | S(CH₂)₂(1-c-Pen)OH |
| 7 | RS | 7-Cl | CH═CH | SCH₂(R)CHMeCO₂H | S(CH₂)₂(1,2-phe)CMe₂OH |
| 8 | S | 7-Cl | C≡C | SCH₂(S)CHMeCO₂H | (CH₂)₂(1,2-phe)CMe₂OH |
| 9 | RS | 7-Cl | CH═CH | SCH₂CHMeCO₂H | (1,4-phe)CMe₂OH |
| 10 | RS | 7-Cl | C≡C | SCH₂CHEtCO₂H | (CH₂)₂(1,2-phe)CMe₂OH |
| 11 | RS | 7-Cl | CH═CH | SCH₂CHEtCO₂H | (1,3-phe)CMe₂OH |
| 12 | S | 7-Cl | CH═CH | SCH₃(S)CHEtCO₂H | (CH₂)₃(1,2-phe)CMe₂OH |
| 13 | RS | 7-Cl | CH═CH | S(CH₂)₂CHMeCO₂H | (CH₂)₂(1,2-phe)CMe₂OH |
| 14 | RS | 7-Cl | C≡C | S(CH₂)₂CO₂H | (CH₂)₂(1,2-phe)CMe₂OH |
| 15 | RS | 7-Cl | CH═CH | SCH₂CHMeCO₂H | (CH₂)₂(1,2-phe)CMe₂OH |
| 16 | S | 7-Cl | CH═CH | SCH₂(S)CHMeCO₂H | (CH₂)₂(1,2-phe)CMe₂OH |
| 17 | R | 7-Cl | CH═CH | SCH₂(S)CHMeCO₂H | (CH₂)₂(1,2-phe)CMe₂OH |
| 18 | S | 7-Cl | CH═CH | S(CH₂)₂CO₂H | S(CH₂)₂CMe₂OH |
| 19 | S | 7-Cl | CH═CH | SCH₂CHMeCO₂H | (CH₂)₂(1,2-phe)C(CF₃)₂OH |
| 20 | RS | 7-Cl | CH═CH | SCH₂CHMeCO₂H | (CH₂)₂(1,3-phe)C(CF₃)₂OH |
| 21 | RS | 7-Cl | CH═CH | SCH₂CHMeCO₂H | (CH₂)₂(1,3-phe)CMe₂OH |
| 22 | RS | 7-Cl | CH═CH | SCH₂CHEtCO₂H | SCH₂CMe₂CMe₂OH |
| 23 | RS | 7-Cl | CH═CH | SCH₂CHMeCMe₂OH | (CH₂)₂(1,2-phe)CO₂H |
| 24 | RS | 7-Cl | CH═CH | SCH₂CHMeCMe₂OH | (CH₂)₂(1,2-phe)CONH₂ |
| 25 | RS | 7-Cl | CH═CH | SCH₂CHMeCO₂H | SCH₂(1,2-phe)CMe₂OH |
| 26 | RS | 7-Cl | CH═CH | SCH₂CHMeCO₂H | (CH₂)₂(1,4-phe)CMe₂OH |
| 27 | RS | 7-Cl | CH═CH | SCH₂CHEtCO₂H | (CH₂)₂(1,2-phe)CMe₂OH |
| 28 | RS | 7-Cl | CH═CH | SCH₂CH(OMe)CO₂H | (CH₂)₂(1,2-phe)CMe₂OH |
| 29 | S | 7-Cl | CH═CH | SCH₂(R)CHEtCO₂H | (CH₂)₂(1,2-phe)CMe₂OH |
| 30 | RS | 7-Cl | CH═CH | S(CH₂)₂CO₂H | (CH₂)₂(1,2-phe)CH(CF₃)OH |
| 31 | S | 7-Cl | CH═CH | SCH₂(R)CHMeCO₂H | (CH₂)₂(1,2-phe)CMe₂OH |
| 32 | S | 7-Cl | CH═CH | SCH₂(S)CHEtCO₂H | (CH₂)₂(1,2-phe)CMe₂OH |
| 33 | RS | 7-Cl | CH═CH | SCH₂CMe₂CO₂H | (CH₂)₂(1,2-phe)CMe₂OH |
| 34 | RS | 7-Cl | CH═CH | SCH₂CHEtCO₂H | (CH₂)₂(1,3-phe)CMe₂OH |
| 35 | RS | 7-Cl | CH═CH | SCH₂CHEtCO₂H | (CH₂)₂(1,2-phe)C(CF₃)₂OH |
| 36 | RS | H | CH═CH | SCH₂CHMeCO₂H | (CH₂)₂(1,2-phe)CMe₂OH |
| 37 | RS | H | CH═CH | SCH₂CHEtCO₂H | (CH₂)₂(1,2-phe)CMe₂OH |
| 38 | RS | 7-Cl | CH═CH | SCH₂CHEtCO₂H | (CH₂)₂(4-Br-1,2-phe)CMe₂OH |
| 39 | RS | 7-Cl | CH═CH | SCH₂CHEtCO₂H | (CH₂)₂(1,2-phe)CMeEtOH |
| 40 | RS | 7-Cl | CH═CH | SCH₂CHEtCO₂H | (CH₂)₂(1,2-phe)CEt₂OH |
| 41 | RS | 7-Cl | CH═CH | SCH₂CHEtCO₂H | (CH₂)₂(1,2-phe)C((CH₂)₃)OH |
| 42 | RS | 7-Cl | CH═CH | SCH₂CHMeCO₂H | (CH₂)₂(1,2-phe)CMe₂NH₂ |
| 43 | RS | 7-Cl | CH═CH | SCH₂CHEtCO₂H | (CH₂)₂(1,2-phe)CHMeNHMe |
| 44 | RS | 7-Cl | CH═CH | SCH₂CHEtCO₂H | (CH₂)₂(1,2-phe)CHMeNMe₂ |
| 45 | RS | 7-Cl | CH═CH | SCH₂CHEtCO₂H | (CH₂)₂(2,5-fur)CMe₂OH |
| 46 | RS | 7-Cl | CH═CH | SCH₂CHEtCO₂H | (CH₂)₂(2,6-pye)CMe₂OH |
| 47 | RS | 7-Cl | CH═CH | SCH₂CHEtCO₂H | (CH₂)₂(4,2-pye)CMe₂OH |
| 48 | RS | 7-Cl | CH═CH | SCH₂CHEtCO₂H | (CH₂)₂(2,5-thio)CMe₂OH |
| 49 | RS | 7-Cl | CH═CH | SCH₂CHEtCO₂H | (CH₂)₂(3,2-pye)CMe₂OH |
| 50 | RS | 7-CN | CH═CH | SCH₂CHEtCO₂H | (CH₂)₂(1,4-phe)CMe₂OH |
| 51 | RS | 7-CF₃ | CH═CH | SCH₂CHEtCO₂H | (CH₂)₂(1,4-phe)CMe₂OH |
| 52 | RS | 7-Cl | CH═CH | SCH₂CHMeCONHS(O)₂Me | (CH₂)₂(1,2-phe)CMe₂OH |
| 53 | RS | 7-NO₂ | CH═CH | SCH₂CHMeCONH₂ | (CH₂)₂(1,2-phe)CMe₂OH |
| 54 | RS | 7-Cl | CH═CH | SCH₂CHMeCONHMe | (CH₂)₂(1,2-phe)CMe₂OH |
| 55 | RS | 7-Cl | CH═CH | SCH₂CHMeTz | (CH₂)₂(1,2-phe)CMe₂OH |
| 56 | RS | 7-Cl | CH═CH | SCH₂CHEtTz | (CH₂)₂(1,2-phe)CMe₂OH |
| 57 | RS | 7-Cl | CH═CH | SCH₂CHEtCONHS(O)₂CF₃ | (CH₂)₂(1,2-phe)CMe₂OH |
| 58 | RS | 7-Cl | CH═CH | SCH₂CHMeNO₂ | (CH₂)₂(1,2-phe)CMe₂OH |
| 59 | RS | 7-Cl | CH═CH | S(CH₂)₂CONHS(O)₂Ph | (CH₂)₂(1,2-phe)CMe₂OH |
| 60 | R | 7-Cl | CH═CH | SCH₂(S)CHEtCO₂H | (CH₂)₂(1,2-phe)CMe₂OH |
| 61 | RS | 7-Cl | CH═CH | S(CH₂)₂CO₃H | (CH₂)₂(1,2-phe)CH₂CMe₂OH |
| 62 | RS | 7-Cl | CH═CH | S(CH₃)₂CMe₂OH | (1,3-phe)CO₂H |
| 63 | RS | 7-Cl | CH═CH | SCH₂CH(n-Pr)CO₂H | (CH₂)₂(1,2-phe)CMe₂OH |
| 64 | RS | 7-Br | CH═CH | SCH₂CHEtCO₂H | (CH₂)₂(1,2-phe)CMe₂OH |
| 65 | S | 7-Cl | CH═CH | SCH₂CH(CH₂CH═CH₂)CO₂H | (CH₂)₂(1,2-phe)CMe₂OH |
| 66 | S | 7-Cl | CH═CH | SCH₂CHEtCO₂H | (CH₂)₂(1,2-phe)CMe₂OH |
| 67 | S | 7-Cl | CH═CH | SCH₂CH(CH₂SMe)CO₂H | (CH₂)₂(1,2-phe)CMe₂OH |
| 68 | S | 7-Cl | CH═CH | SCH₂CH(c-Pr)CO₂H | (CH₂)₂(1,2-phe)CMe₂OH |
| 69 | S | 7-Cl | CH═CH | SCH₂CH(CH₂C═CH)CO₂H | (CH₂)₂(1,2-phe)CMe₂OH |
| 70 | S | 7-Cl | CH═CH | SCH₂CH(CH₂Ph)CO₂H | (CH₂)₂(1,2-phe)CMe₂OH |
| 71 | RS | 7-Cl | CH═CH | SCH₂CHMeCO₂H | (CH₂)₂(1,2-phe)CHMeOH |
| 72 | S | 7-Cl | CH═CH | SCH₂CHPhCO₂H | (CH₂)₂(1,2-phe)CMe₂OH |
| 73 | S | 7-Cl | CH═CH | SCH₂(S)CHEtCO₂H | (CH₂)₂(1,2-phe)CH₂CMe₂OH |
| 74 | S | 7-Cl | CH═CH | SCH₂CHEtCO₂H | (CH₂)₂(1,2-phe)CH₂CHMeOH |
| 75 | S | 7-Cl | CH═CH | SCH₂CH(n-Pr)CO₂H | (CH₂)₂(1,2-phe)CHMeOH |
| 76 | RS | 7-Cl | CH═CH | SCH₂CHEtCO₂H | (1,2-phe)CMe₂OH |
| 77 | S | 7-Cl | CH═CH | SCH₂CHEtCO₂H | (CH₂)₂(1-2-phe)C(CH₂OCH₂)OH |
| 78 | RS | 7-Cl | CH═CH | S(CH₂)₂CMe₂OH | (CH₂)₂(1,2-phe)CO₂H |
| 79 | S | 7-Br | CH═CH | SCH₂(S)CHEtCO₂H | (CH₂)₂(1,2-phe)CMe₂OH |
| 80 | S | 7-Cl | CH═CH | S(CH₂)₂CMe₂OH | (CH₂)₂(1,2-phe)CHMeCO₂H |
| 81 | RS | 7-Cl | CH═CH | S(CH₂)₂CO₂H | CH₂CHOH(1,4-phe)CN |
| 82 | RS | 7-Cl | CH═CH | S(CH₂)₂CO₂H | CH₂CHOH(1,3-phe)CN₄H |
| 83 | RS | 7-Cl | CH═CH | S(CH₂)₂CO₂H | CH₂CHOH(1,4-phe)CN₄H |
| 84 | S | 7-Cl | CH═CH | S(CH₂)₂CO₂H | (CH₂)₂(1,2-phe)CMe₂OH |
| 85 | S | 7-Cl | CH═CH | SCH₂CHCF₃CO₂H | (CH₂)₂(1,2-phe)CMe₂OH |
| 86 | S | 7-Cl | CH═CH | S(CH₂)₃CO₂H | (CH₂)₂(1,2-phe)CMe₂OH |
| 87 | S | 7-Cl | CH═CH | S(CH₂)₂CHMeCO₂H | (CH₂)₂(1,2-phe)CMe₂OH |
| 88 | S | 7-Cl | CH═CH | S(O)₂CH₂(S)CHEtCO₂H | (CH₂)₂(1,2-phe)CMe₂OH |
| 89 | S | 7-Cl | CH═CH | SCH₂CH(CH₂OMe)CO₂H | (CH₂)₂(1,2-phe)CMe₂OH |
| 90 | S | 7-Cl | CH═CH | S(CH₂)₂OMe₂OH | (CH₂)₂(1,2-phe)CO₂H |
| 91 | R | 7-Cl | CH═CH | S(CH₂)₂CMe₂OH | (CH₂)₂(1,2-phe)CO₂H |
| 92 | S | 7-Cl | CH═CH | SCH₂(S)CHEtCO₂H | (CH₂)₂(1,3-phe)CMe₂OH |
| 93 | S | 7-Cl | CH═CH | SCH₂CHEtCO₂H | (CH₂)₂(1,3-phe)(1,1-c-Be)OH |
| 94 | S | 7-Cl | CH═CH | S(CH₂)₂CMe₂OH | (CH₂)₃(1,2-phe)COOH |
| 95 | R | 7-Cl | CH═CH | S(CH₂)₂CO₂H | S(CH₂)₂(1,1-c-Pen)OH |
| 96 | S | 7-Cl | CH═CH | SCH₂CH(CH₂CF₃)CO₂H | (CH₂)₂(1,2-phe)CMe₂OH |
| 97 | S | 7-Cl | CH═CH | S(CH₂)₂CMe₂OH | (CH₂)₂(4-Cl-1,2-phe)CO₂H |
| 98 | S | 7-Cl | CH═CH | SCH₂CH(n-Pr)CO₂H | (CH₂)₂(1,2-phe)CMe₂OH |
| 99 | R | 7-Cl | CH═CH | SCH₂(S)CHEtCONHS(O)₂Me | (CH₂)₂(1,2-phe)CMe₂OH |
| 100 | S | 7-Cl | CH═CH | S(CH₂)₂CMeOH | (CH₂)₂(1,3-phe)CMe₂CO₂H |
| 101 | S | 7-Cl | CH═CH | S(CH₂)₂CMeOH | (CH₂)₂(1,3-phe)CHMeCO₂H |
| 102 | S | 7-Cl | CH═CH | S(CH₂)₃CMe₂OH | (CH₂)₂(1,2-phe)CO₂H |
| 103 | S | 7-Cl | CH═CH | SCH₂(S)CHEtCO₂H | (CH₂)₂(1,4-phe)CMe₂OH |
| 104 | RS | 7-Cl | CH═CH | S(CH₂)₂CMe₂OH | (CH₂)₂(1,3-phe)CN₄H |
| 105 | S | 7-Cl | CH═CH | S(CH₂)₂CMe₂OH | (CH₂)₂(1,2-phe)CHMeCO₂H |
| 106 | S | 7-Cl | CH═CH | S(CH₂)₃CMe₂OH | (CH₂)₂(1,2-phe)CHMeCONHS(O)₂CH₃ |
| 107 | S | 7-Cl | CH═CH | S(CH₂)₂CMe₂OH | (CH₂)₃(1,2-phe)CO₂H |
| 108 | R | 7-Cl | CH═CH | S(O)₂CH₂(S)CHEtCO₂H | (CH₂)₂(1,2-phe)CMe₂OH |
| 109 | S | 7-Cl | CH═CH | S(CH₂)₂CMe₂OH | (CH₂)₂(4-Cl-1,2-phe)CHMeCO₂H |
| 110 | S | 7-Cl | CH═CH | SCH₂(S)CHMeCO₂H | (CH₂)₂(1,2-phe)CH₂CMe₂OH |
| 111 | S | 7-Cl | CH═CH | S(CH₂)₂CMe₂OH | (CH₂)₂(1,2-phe)CO₂Me |
| 112 | S | 7-Cl | CH═CH | S(CH₂)₃CMe₂OH | (CH₂)₂(4-Cl-1,2-phe)CO₂H |
| 113 | R | 7-Cl | CH═CH | S(CH₂)₃CMe₂OH | (CH₂)₃(4-Cl-1,2-phe)CO₂H |
| 114 | S | 7-Cl | CH═CH | S(CH₂)₃CMe₂OH | (CH₂)₂(1,2-phe)CMe₂CO₂H |
| 115 | S | 7-Cl | CH═CH | S(CH₂)₂CMe₂OH | (CH₂)₃(R)CHMe₂CO₂H |
| 116 | S | 7-Cl | CH═CH | S(CH₂)₃CEt₂OH | (CH₂)₂(1,2-phe)CO₂H |
| 117 | S | 7-Cl | CH═CH | S(CH₂)₃CEt₂OH | (CH₂)₂(1,2-phe)CHMeCO₂H |
| 118 | R | 7-Cl | CH═CH | SCHMeCH₂CO₂H | (CH₂)₂(1,2-phe)CMe₂OH |
| 119 | S | 7-Cl | CH═CH | S(CH₂)₃CMe₂OH | (CH₂)₂(1,2-phe)CHEtCO₂H |
| 120 | S | 7-Cl | CH═CH | S(CH₂)₃CMe₂OH | (CH₂)₂(1,2-phe)CH(n-Pr)CO₂H |
| 121 | S | 7-Cl | CH═CH | S(CH₂)₃CMe₂OH | (CH₂)₂(1,2-phe)CH(i-Pr)CO₂H |
| 122 | R | 7-Cl | CH═CH | SCH₂MeCHMeCO₂H | (CH₂)₂(1,2-phe)CMe₂OH |
| 123 | R | 7-Cl | CH═CH | S(CH₂)₂CMe₂OH | (CH₂)₃(R)CHMeCO₂H |
| 124 | R | 7-Cl | CH═CH | SCH₂(S)CHMeCN₄H | (CH₂)₂(1,2-phe)CMe₂OH |
| 125 | S | 7-Cl | CH═CH | SCH₂(S)CHMeCO₂H | (CH₂)₂(3-OH-1,4-phe)CHMeOH |
| 126 | S | 7-Cl | CH═CH | S(CH₂)₃CHMeOH | (CH₂)₂(1,2-phe)CHMeCO₂H |
| 127 | R | 7-Cl | CH═CH | S(S)CHMeCH₂CO₂H | (CH₂)₂(1,2-phe)CMe₂OH |
| 128 | R | 7-Cl | CH═CH | S(R)CHMeCH₂CO₂H | (CH₂)₂(1,2-phe)CMe₂OH |
| 129 | R | 7-Cl | CH═CH | S(S)CHMe(S)CHMeCO₂H | (CH₂)₂(1,2-phe)CMe₂OH |
| 130 | R | 7-Cl | CH═CH | S(R)CHMe(R)CHMeCO₂H | (CH₂)₂(1,2-phe)CMe₂OH |
| 131 | R | 7-Cl | CH═CH | SCHEtCH₂CO₂H | (CH₂)₂(1,2-phe)CMe₂OH |
| 132 | S | 7-Cl | CH═CH | S(CH₂)₃CHMeOH | (CH₂)₂(1,2-phe)CHEtCO₂H |
| 133 | S | 7-Cl | CH═CH | SCH₂(S)CHMeOC₂H | (CH₂)₂(4-OMe-1,2-phe)CMe₂CO₂H |
| 134 | R | 7-Cl | CH═CH | SCMe₂CH₂CO₂H | (CH₂)₂(1,2-phe)CMe₂OH |
| 135 | R | 7-Cl | CH═CH | SCH₂CHMeCH₂CO₂H | (CH₂)₂(1,2-phe)CMe₂OH |
| 136 | R | 7-CF₃ | CH═CH | SCH₂CMe₂CH₂CO₂H | (CH₂)₂(1,2-phe)CMe₂OH |
| 137 | S | 7-CN | CH═CH | SCH₂CMe₂CH₂CO₂H | (CH₂)₂(1,2-phe)CO₂H |
| 138 | S | 7-Cl | CH═CH | S(CH₂)₃CMe₂OH | (CH₂)₂(1,2-phe)(R)CHEtCO₂H |
| 139 | S | 7-Cl | CH═CH | S(CH₂)₃CMe₂OH | (CH₂)₂(1,2-phe)((S)CHEtCO₂H |
| 140 | S | 7-Cl | CH═CH | S(CH₂)₃CMe₂OH | (CH₂)₂(4-Cl-1,2-phe)CHEtCO₂H |
| 141 | S | 7-Cl | CH═CH | S(CH₂)₃CMe₂OH | (CH₂)₂(1,2-phe)CEt₂CO₂H |
| 142 | S | 7-Cl | CH═CH | S(CH₂)₃CMe₂OH | (CH₂)₂(1,2-phe)CH₂CO₂H |
| 143 | S | 7-Cl | CH═CH | S(CH₂)₃CMe₂OH | (CH₂)₂(1,2-phe)CH(OH)CO₂H |
| 144 | S | 7-Cl | CH═CH | S(CH₂)₃CMe₂OH | (CH₂)₂(1,2-phe)CHEtCO₂H |
| 145 | S | 7-Cl | CH═CH | S(CH₂)₃CMe₃OH | (CH₂)₂CHMeCH₂CO₂H |
| 146 | R | 7-Cl | CH═CH | SCH₂CMe₂CH₂CO₂H | (CH₂)₂(1,2-phe)CMe₂OH |
| 147 | S | 7-Cl | CH═CH | S(CH₂)₄CMe₂OH | (CH₂)₂(1,2-phe)CHEtCO₂H |
| 148 | S | 6-Cl | CH═CH | S(CH₂)₃CMe₂OH | (CH₂)₂(1,2-phe)CO₂H |
| 149 | S | 8-Cl | CH═CH | S(CH₂)₃CMe₂OH | (CH₂)₂(1,2-phe)CO₂H |
| 150 | S | 7-F | CH═CH | S(CH₂)₃CMe₂OH | (CH₂)₂(1,2-phe)CHEtCO₂H |
| 151 | S | 7-Br | CH═CH | S(CH₂)₃CMe₂OH | (CH₂)₂(1,2-phe)CHMeCO₂H |
| 152 | S | 7-I | CH═CH | SCH₂C(1,1-c-Pr)CH₂CO₂H | (CH₂)₂(1,2-phe)CMe₂OH |
| 153 | S | 7-NO₂ | CH═CH | SCH₂C(1,1-c-Pr)CH₂CO₂H | (CH₂)₂(1,2-phe)CMe₂OH |
| 154 | R | 7-N₃ | CH═CH | SCH₂C(1,1-c-Pr)CH₂CO₂H | (CH₂)₂(1,2-phe)CMe₂OH |
| 155 | RS | 7-Cl | CH═CH | S(CH₂)₂CMe₂OH | (CH₂)₂CMe₂CH₂CO₂H |
| 156 | R | 7-Cl | CH═CH | S(1,2-phe)CH₂CO₂H | (CH₂)₂(1,2-phe)CMe₂OH |
| 157 | R | 7-Cl | CH═CH | S(CH₂)₃CMe₂OH | (CH₂)₂(1,2-phe)CHEtCO₂H |
| 158 | S | 7-Cl | CH═CH | S(CH₂)₂CMe₂OH | (CH₂)₂(1,2-phe)CHEtCO₂H |
| 159 | S | 7-Cl | CH═CH | S(CH₂)₃CMe(4-Cl—Ph)OH | (CH₂)₂(1,2-phe)CHEtCO₂H |
| 160 | R | 7-Cl | CH═CH | SCH₂(1,2-phe)CMe₂OH | (CH₂)₂CMe₂CH₂CO₂H |
| 161 | R | 7-Cl | CH═CH | SCH₂(1,1-c-Pr)CH₂CO₂H | (CH₂)₂(1,2-phe)CMe₂OH |
| 162 | R | 7-Cl | CH═CH | SCH₂(1,1-c-Bu)CH₂CO₂H | (CH₂)₂(1,2-phe)CMe₂OH |
| 163 | R | 7-Cl | CH═CH | SCH₂CMe₂CHMeCO₂H | (CH₂)₂(1,2-phe)CMe₂OH |
| 164 | S | 7-Cl | CH═CH | SCH₂(1,2-phe)CMe₂OH | (CH₂)₂CMe₂CH₂CO₂H |
| 165 | R | 7-Cl | CH═CH | SCHMeCMe₂CH₂CO₂H | (CH₂)₂(1,2-phe)CMe₂OH |
| 166 | R | 7-Cl | CH═CH | S(1,1-c-Pr)CH₂CO₂H | (CH₂)₂(1,2-phe)CMe₂OH |
| 167 | R | 7-Cl | CH═CH | S(1,1-c-Pr)CHMeCO₂H | (CH₂)₂(1,2-phe)CMe₂OH |

The LT antagonist compounds for use with present invention may be made as disclosed in U.S. Pat. No. 5,565,473, incorporated herein by reference.

### The Salts

The pharmaceutical compositions of the present invention comprise a compound for use with the present invention as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary, and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

It will be understood that in the discussion of methods of treatment which follows, references to the compounds of Formulas disclosed herein are meant to also include the pharmaceutically acceptable salts.

For example, an example of a salt of Formula II is:

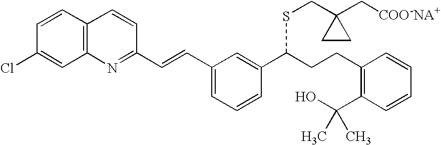

### Dose Ranges

The magnitude of prophylactic or therapeutic dose of a compound of the present invention will, of course, vary with the nature of the severity of the condition to be treated and with the particular compound of the present invention and its route of administration. It will also vary according to the age, weight and response of the individual patient. In general, the daily dose range lie within the range of from about 0.001 mg to about 100 mg per kg body weight of a mammal, preferably 0.01 mg to about 10 mg per kg, and most preferably 0.1 to 1 mg per kg, in single or divided doses. On the other hand, it may be necessary to use dosages outside these limits in some cases.

For use where a composition for intravenous administration is employed, a suitable dosage range is from about 0.001 mg to about 25 mg (preferably from 0.01 mg to about 1 mg) of a compound of the present invention per kg of body weight per day and for cytoprotective use from about 0.1 mg to about 100 mg (preferably from about 1 mg to about 100 mg and more preferably from about 1 mg to about 10 mg) of a compound of the present invention per kg of body weight per day.

In the case where an oral composition is employed, a suitable dosage range for is, e.g. from about 0.01 mg to about 100 mg of a compound of the present invention per kg of body weight per day, preferably from about 0.1 mg to about 10 mg per kg and for cytoprotective use from 0.1 mg to about 100 mg (preferably from about 1 mg to about 100 mg and more preferably from about 10 mg to about 100 mg) of a compound of the present invention per kg of body weight per day. Of course, the dose may vary at the discretion of one of ordinary skill in the art.

### Pharmaceutical Compositions

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. They may be conveniently presented in unit dosage form and prepared by any of the methods well-known in the art of pharmacy.

The pharmaceutical compositions of the present invention comprise a compound of the present invention as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

For administration by inhalation, the compounds of the present invention are conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery system for inhalation is a metered dose inhalation (MDI) aerosol, which may be formulated as a suspension or solution of compound I in suitable propellants, such as fluorocarbons or hydrocarbons.

Suitable topical formulations of the present invention include transdermal devices, aerosols, creams, ointments, lotions, dusting powders, and the like.

In practical use, the compounds of the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of the present invention may also be administered by controlled release means and/or delivery devices such as those described in U.S. Pat. Nos. 3,845,770; 3,916,899; 3,536,809; 3,598,123; 3,630,200 and 4,008,719, the disclosures of which are hereby incorporated herein by reference.

The pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 2.5 mg to about 500 mg of the active ingredient and each cachet or capsule contains from about 2.5 to about 500 mg of the active ingredient.

The following are examples of representative pharmaceutical dosage forms for the compounds of the present invention:

| Injectable Suspension (I.M.) | mg/ml | |
|---|---|---|
| Compound of Formula I | 10 | |
| Methylcellulose | 5.0 | |
| Tween 80 | 0.5 | |
| Benzyl alcohol | 9.0 | |
| Benzalkonium chloride | 1.0 | |
| Water for injection to a total volume of 1 ml | | |
| Tablet | mg/tablet | |
| Compound of Formula I | 25 | |
| Microcrystalline Cellulose | 415 | |
| Providone | 14.0 | |
| Pregelatinized Starch | 43.5 | |
| Magnesium Stearate | 2.5 | |
| | 500 | |
| Capsule | mg/capsule | |
| Compound of Formula I | 25 | |
| Lactose Powder | 573.5 | |
| Magnesium Stearate | 1.5 | |
| | 600 | |
| Aerosol | Per canister | |
| Compound of Formula I | 24 | mg |
| Lecithin, NF Liquid Concentrate | 1.2 | mg |
| Trichlorofluoromethane, NF | 4.025 | gm |
| Dichlorodifluoromethane, NF | 12.15 | gm |

As indicated above, the magnitude of prophylactic or therapeutic dose of a compound of the present invention will, of course, vary with the nature of the severity of the condition to be treated and with the particular compound of the present invention and its route of administration. It will also vary according to the age, weight and response of the individual patient, as determined by one of ordinary skill in the art.

In general, one of the primary initial goals of such drug therapy is to establish a daily oral dosage, so that a single convenient “unit dosage” formulation (usually a pill, such as a tablet, capsule, etc.) can be taken by a patient each day. The dosage levels that have already been established for the anti-asthma formulations of zafirlukast (“Accolate”, which normally is taken twice a day) and montelukast (“Singulair”, which normally is taken once a day) offer a good starting point to one of ordinary skill for evaluating preferred dosages that will have maximum beneficial effects in preventing migraine headaches. Evaluative tests to optimize the daily dosages for various patients with particular migraine patterns or severities can be carried out using no more than routine experimentation.

Two or more LT antagonist drugs can be provided in a single formulation, if desired. For example, a first LT antagonist can be used which blocks a first specific type of LT receptor, and a second LT antagonist can be used which blocks a second specific type of LT receptor. Alternately or additionally, a first LT antagonist which inhibits leukotriene biosynthesis can be included in a formulation with a second LT antagonist which suppresses activity at one or more LT receptor types.

In another embodiment of the present invention, a leukotriene receptor antagonist compound or composition described here in is administered in combined therapy with a nasal steroid.

In embodiments the steroid is intranasal budesonide. For example, the intranasal budesonide may be Rhinocort AQ, available from Astra Zeneca, Wilmington, Del. See U.S. Pat. Nos. 6,686,346, 6,291,445, and 3,992,534, all incorporated herein by reference.

Other examples of the steroids include corticosteroids that have previously administered by intranasal administration may be used, such as beclomethasone (Vancenase® or Beconase®), flunisolide (Nasalide®)), fluticasone proprionate (Flonase®), triamcinolone acetonide (Nasacort®), loterednol etabonate (Locort®) and mometasone (Nasonex®. See U.S. Patent Application Publication 20050227297 for examples of corticosteroids of the present invention.

This method additionally results in normalization of residual sleep-disordered breathing after tonsillectomy and adenoidectomy.

A method of these embodiments is in Kheirandish et al., Intranasal Steroids and Oral Leukotriene Modifier Therapy in residual Sleep-Disordered Breathing After Tonsillectomy and Adenoidectomy in Children, Pediatrics 2006; 117; 61-66, incorporated herein by reference in its entirety.

As shown in the article incorporated herein by reference, an example of this embodiment demonstrates that a 12-week course of an orally administered leukotriene receptor antagonist combined with intranasal administration of a corticosteroid is associated with improvements in upper airway patency and in the severity of SDB that occurred after T&A in children and that these improvements fail to occur when no treatment is administered.

### EXAMPLE

The efficacy of leukotriene antagonists as a treatment for snoring and sleep apnea is assessed by administering an appropriate pharmaceutical composition thereof to patients suffering from snoring and sleep apnea for a treatment period and collecting data from the patient before and after the treatment period. Specifically, the patients undergo overnight polysomnography before and after the treatment period. Polysomnography is the monitoring of relevant normal and abnormal physiological activity during sleep and involves collecting measurement, including the following:

(1) Snoring Score—a measurement of the severity and loudness of snoring on a scale from 0-8, the higher the score, the more severe and loud the snoring;

(2) Apnea Hypopnea Index (AHI)—a measurement of the number of apneic (cessation of breathing) and hypopneic (abnormal decrease in the depth and rate of breathing) episodes combined per hour of sleep;

(3) Respiratory Arousal Index—a measurement of sleep fragmentation characterized by the number of respiratory or snoring-associated arousals combined per hour of sleep; and

(4) Adenoid Size—a measurement of the ability of air to flow through the airway as assessed by taking a lateral film of the neck of the patient and expressing the size of the adenoid as a percentage of the patients total airway size.

The mean measurements taken from the patients, before and after the treatment, are shown in Table 2.

TABLE 2

| | Before Treatment | After Treatment |
|---|---|---|
| Snoring Score | 6.4 ± 2.2 | 2.7 ± 1.0 |
| (on 0-8 Scale) | | |
| Apnea Hypopnea Index | 3.6 ± 1.3 | 2.1 ± 0.8 |
| (# per hour total sleep time) | | |
| Respiratory Arousal Index | 7.8 ± 1.7 | 3.3 ± 0.9 |
| (# per hour total sleep time) | | |
| Adenoid Size | 68.3% ± 4.7 | 45.6% ± 3.9 |
| (% of total airway size) | | |

As shown by the measurements in Table 2, leukotriene antagonists are effective in the treatment of snoring and sleep apnea. Specifically, in response to treatment therewith, the severity and loudness of snoring is decreased, of the number of apneic and hypopneic episodes are decreased, sleep fragmentation is decreased, and the size of the adenoids is decreases, allowing air to flow more readily through the airway.

Although it is not necessary, it is preferred that the leukotriene antagonist in its appropriate pharmaceutical composition be administered for a treatment period of 8 or more weeks, wherein the appropriate dose of the composition is administered once daily. The treatment period may be continuous.

One of the primary advantages of using LT antagonist drugs in connection with the present invention is that such drugs apparently do not create any problems of tolerance or dependency. Instead, these drugs appear to help suppress, control, and reduce, over the long term, the gradually cumulative problems of the instant indications. Accordingly, LT antagonists appear to offer an ideal approach to a long-term preventive (“prophylactic”) treatment.

As a best mode of the present invention, a pharmaceutical composition of Formula II is administered in an known amount (such as those used for the treatment of asthma) to relieve or prevent indications associated with sleep apnea and/or snoring.

It will be obvious to those skilled in the art that further modifications may be made to the embodiments described herein without departing from the spirit and scope of the present invention. Other embodiments in the invention will be apparent to those skilled in the art from consideration of the specification and the practice of the invention as disclosed herein. The aforementioned preferred embodiments are for exemplary purposes, not intended to limit the spirit and scope of the present invention.

Unless otherwise indicated, all numbers expressing quantities of ingredients, properties such as reaction conditions, and so forth used in the Specification and Claims are to be understood as being modified in all instances by the term “about.” Accordingly, unless indicated to the contrary, the numerical parameters set forth in the Specification and Claims are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references are incorporated herein by reference.

I claim:

1. A method for treating at least one of snoring and sleep apnea in a mammal using a leukotriene antagonist, comprising the steps of:
providing a pharmaceutical composition of the leukotriene antagonist; and
administering an effective amount of the pharmaceutical composition to the mammal for a treatment period such that there is a reduction in the size of the adenotonsillar tissue of the mammal relative to the airway of the mammal.

2. The method of claim 1, wherein the leukotriene antagonists is selected from the following formula: 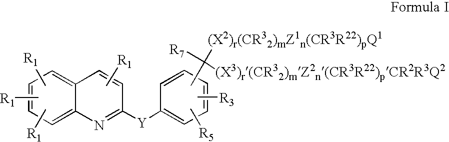
wherein:
R₁ is H, halogen, —CF₃, —CN, —NO₂, or N₃;
R₂ is lower alkyl, lower alkenyl, lower alkynyl, —CF₃, —CH₂F, —CH₂F₂, CH₂CF₃, substituted or unsubstituted phenyl, substituted or unsubstituted benzyl, substituted or unsubstituted 2-phenethyl, or two R² groups joined to the same carbon may form a ring of up to 8 members containing 0-2 heteroatoms chosen from O, S, and N;
R³ is H or R²;
CR³R²² may be the radical of a standard amino acid;
R⁴ is halogen, —NO₂, —CN, —OR₃, —SR³, NR³R³, NR³C(O)R⁷ or R³;
R⁵ is H, halogen, —NO₂, —N₃, —CN, —SR², —NR³R³, —OR³, lower alkyl, or —C(O)R³;
R⁶ is (CH₂)ₛ—C(R⁷R⁷)—(CH₂)ₛ—R⁸ or —CH₂C(O)NR¹²R¹²;
R⁷ is H or C₁₋₄ alkyl;
R⁸ is
A) a monocyclic or bicyclic heterocyclic radical containing from 3 to 12 nuclear carbon atoms and 1 or 2 nuclear heteroatoms selected from N, S or O and with each ring in the heterocyclic radical being formed of 5 or 6 atoms, or
B) the radical W—R⁹;
R⁹ contains up to 20 carbon atoms and is (1) an alkyl group or (2) an alkylcarbonyl group of an organic acyclic or monocyclic carboxylic acid containing not more than 1 heteroatom in the ring;
R¹⁰ is —SR¹¹, —OR¹² or —NR¹²R¹²;
R¹¹ is lower alkyl, —C(O)R¹⁴, unsubstituted phenyl, or unsubstituted benzyl;
R¹² is H, R¹¹ or two R¹² groups joined to the same N may form a ring of 5 or 6 members containing 1-2 heteroatoms chosen from O, S, and N;
R¹³ is lower alkyl, lower alkenyl, lower alkynyl, —CF₃ or substituted or unsubstituted phenyl, benzyl, or 2-phenethyl;
R¹⁴ is H or R¹³;
R¹⁶ is H, C₁₋₄ alkyl, or OH;
R¹⁷ is lower alkyl, lower alkenyl, lower alkynyl, or substituted or unsubstituted phenyl, benzyl, or 2-phenethyl;
R¹⁸ is lower alkyl, lower alkenyl, lower alkynyl, —CF₃ or substituted or unsubstituted phenyl, benzyl, or 2-phenethyl;
R¹⁹ is lower alkyl, lower alkenyl, lower alkynyl, —CF₃ or substituted or unsubstituted phenyl, benzyl, or 2-phenethyl;
R²⁰ is H, C₁₋₄ alkyl, substituted or unsubstituted phenyl, benzyl, phenethyl, or pyridinyl or two
R²⁰ groups joined to the same N may form a saturated ring of 5 or 6 members containing 1-2 heteroatoms chosen from O, S, and N;
R²¹ is H or R¹⁷;
R²² is R⁴, CHR⁷OR³, or CHR⁷SR²;
m and m′ are independently 0-8;
n and m′ are independently 0 or 1;
p and p′ are independently 0-8;
m+n+p is 1-10 when r is 1 and X² is O, S, S(O), or S(O)₂;
m+n+p is 0-10 when r is 1 and X² is CR³R¹⁶;
m+n+p is 0-10 when r is O;
m′+m′+p′ is 0-10;
r and r′ are independently 0 or 1;
s is 0-3;
Q¹ is —C(O)OR³, 1H(or 2H)-tetrazol-5-yl, —C(O)OR⁶, —C(O)NHS(O)₂R¹³, —CN, —C(O)NR¹²R¹², —NR²¹S(O)₂R¹², —CN, —NR¹²C(O)NR¹²R¹², —NR²¹C(O)R¹⁸, —OC(O)NR¹²R¹², —C(O)R¹⁹, —S(O)R¹⁸, —S(O)₂R¹⁸, —S(O)₂NR¹²R¹², —NO₂, —NR²¹C(O)OR¹⁷, —C(NR¹²R¹²)═NR¹², —C(R¹³)═NOH; or if Q¹ —C(O)OH and R²² is —OH, —SH, —CHR⁷ OH or —NHR³, then Q¹ and R²² and the carbons through which they are attached may form a heterocyclic ring by loss of water;
Q² is OH or NR²⁰R²⁰;
W is O, S, or NR³;
X² and X³ are independently O, S, S(O), S(O)₂, or CR³R¹⁶;
Y is —CR³═CR³— or —C═C—;
Z¹ and Z² are independently —HET(—R³—R⁵)—;
HET is the diradical of a benzene, a pyridine, a furan, or a thiophene;
and stereoisomers, analogs, and pharmaceutically acceptable salts thereof.

3. The method of claim 1, wherein the leukotriene antagonists is selected from the following formula: 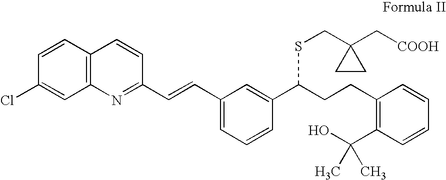
and stereoisomers, analogs, and pharmaceutical salts thereof.

4. The method of claim 1, wherein the leukotriene antagonists is selected from the following formula: 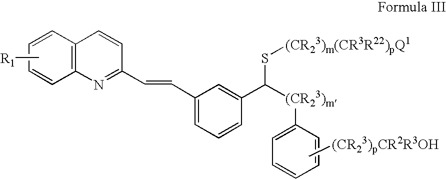
wherein:
R¹H, halogen, CF₃, or CN;
R²² is R³, —CH₂O₃, or —CH₂SR²;
Q¹ is —C(O)OH, 1H(or 2H)-tetrazol-5-yl, —C(O)NHS(O)₂R¹³, —C(O)NR¹²R¹², or —NHS(O)₂R¹³;
m′is 0, 1, 2 or 3;
p′ is 0 or 1;
m+p is 1-5;
the remaining definitions are as in Formula I;
and stereoisomers, analogs, and pharmaceutically acceptable salts thereof.

5. The method of claim 1, wherein the leukotriene antagonists is selected from the following formula: 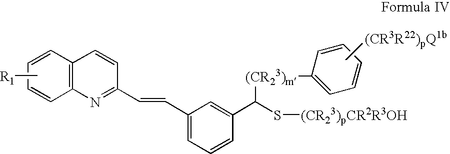
wherein:
R¹ is H, halogen, CF₃, or CN;
R²² is R³, —CH₂O₃, or —CH₂SR²;
Q¹ is —C(O)OH, 1H(or 2H)-tetrazol-5-yl, —C(O)NHS(O)₂R¹³, —C(O)NR¹²R¹², or —NHS(O)₂R¹³;
m′is 0, 1, 2 or 3;
p is 0 or 1
p′ 1-4;
m+p is 0-4;
the remaining definitions are as in Formula I;
and the pharmaceutically acceptable salts thereof.

6. The method of claim 1, wherein the leukotriene antagonist is selected from the group consisting of zafirlukast, montelukast, pranlukast, BAYx7195, LY293111, ICI 204,219, and ONO-1078.

7. A method for treating at least one of snoring and sleep apnea in a mammal using a leukotriene antagonist, comprising the steps of:
providing a pharmaceutical composition of the leukotriene antagonist; and
administering an effective amount of the pharmaceutical composition to a mammal in need thereof.

8. The method of claim 7, wherein the leukotriene antagonists is selected from the following formula: 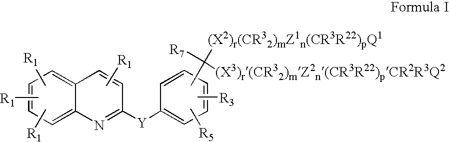
wherein:
R₁ is H, halogen, —CF₃, —CN, —NO₂, or N₃;
R₂ is lower alkyl, lower alkenyl, lower alkynyl, —CF₃, —CH₂F, —CH₂F₂, CH₂CF₃, substituted or unsubstituted phenyl, substituted or unsubstituted benzyl, substituted or unsubstituted 2-phenethyl, or two R² groups joined to the same carbon may form a ring of up to 8 members containing 0-2 heteroatoms chosen from O, S, and N;
R³ is H or R²;
CR³R²² may be the radical of a standard amino acid;
R⁴ is halogen, —NO₂, —CN, —OR₃, —SR³, NR³R³, NR³C(O)R⁷ or R³;
R⁵ is H, halogen, —NO₂, —N₃, —CN, —SR², —NR³R³, —OR³, lower alkyl, or —C(O)R³;
R⁶ is (CH₂)ₛ—C(R⁷R⁷)—(CH₂)ₛ—R⁸ or —CH₂C(O)NR¹²R¹²;
R⁷ is H or C₁₋₄ alkyl;
R⁸ is
A) a monocyclic or bicyclic heterocyclic radical containing from 3 to 12 nuclear carbon atoms and 1 or 2 nuclear heteroatoms selected from N, S or O and with each ring in the heterocyclic radical being formed of 5 or 6 atoms, or
B) the radical W—R⁹;
R⁹ contains up to 20 carbon atoms and is (1) an alkyl group or (2) an alkylcarbonyl group of an organic acyclic or monocyclic carboxylic acid containing not more than 1 heteroatom in the ring;
R¹⁰ is —SR¹¹, —OR¹², or —NR¹²R¹²;
R¹¹ is lower alkyl, —C(O)R¹⁴, unsubstituted phenyl, or unsubstituted benzyl;
R¹² is H, R¹¹ or two R¹² groups joined to the same N may form a ring of 5 or 6 members containing 1-2 heteroatoms chosen from O, S, and N;
R¹³ is lower alkyl, lower alkenyl, lower alkynyl, —CF₃ or substituted or unsubstituted phenyl, benzyl, or 2-phenethyl;
R¹⁴ is H or R¹³;
R¹⁶ is H, C₁₋₄ alkyl, or OH;
R¹⁷ is lower alkyl, lower alkenyl, lower alkynyl, or substituted or unsubstituted phenyl, benzyl, or 2-phenethyl;
R¹⁸ is lower alkyl, lower alkenyl, lower alkynyl, —CF₃ or substituted or unsubstituted phenyl, benzyl, or 2-phenethyl;
R¹⁹ is lower alkyl, lower alkenyl, lower alkynyl, —CF₃ or substituted or unsubstituted phenyl, benzyl, or 2-phenethyl;
R²⁰ is H, C₁₋₄ alkyl, substituted or unsubstituted phenyl, benzyl, phenethyl, or pyridinyl or two
R²⁰ groups joined to the same N may form a saturated ring of 5 or 6 members containing 1-2 heteroatoms chosen from O, S, and N;
R²¹ is H or R¹⁷;
R²² is R⁴, CHR⁷OR³, or CHR⁷SR²;
m and m′ are independently 0-8;
n and m′ are independently 0 or 1;
p and p′ are independently 0-8;
m+n+p is 1-10 when r is 1 and X² is O, S, S(O), or S(O)₂;
m+n+p is 0-10 when r is 1 and X² is CR³R¹⁶;
m+n+p is 0-10 when r is O;
m′+m′+p′ is 0-10;
r and r′ are independently 0 or 1;
s is 0-3;
Q¹ is —C(O)OR³, 1H(or 2H)-tetrazol-5-yl, —C(O)OR⁶, —C(O)NHS(O)₂R¹³, —CN, —C(O)NR¹²R¹², —NR²¹S(O)₂R¹², —CN, —NR¹²C(O)NR¹²R¹², —NR²¹C(O)R¹⁸, —OC(O)NR¹²R¹², —C(O)R¹⁹, —S(O)R¹⁸, —S(O)₂R¹⁸, —S(O)₂NR¹²R¹², —NO₂, —NR²¹C(O)OR¹⁷, —C(NR¹²R¹²)═NR¹², —C(R¹³)═NOH; or if Q¹—C(O)OH and R²² is —OH, —SH, —CHR⁷ OH or —NHR³, then Q¹ and R²² and the carbons through which they are attached may form a heterocyclic ring by loss of water;
Q² is OH or NR²⁰ OR²⁰;
W is O, S, or NR³;
X² and X³ are independently O, S, S(O), S(O)₂, or CR³R¹⁶;
Y is —CR³═CR³— or —C═C—;
Z¹ and Z² are independently —HET(—R³—R⁵)—;
HET is the diradical of a benzene, a pyridine, a furan, or a thiophene;
and stereoisomers, analogs, and pharmaceutically acceptable salts thereof.

9. The method of claim 7, wherein the leukotriene antagonists is selected from the following formula: 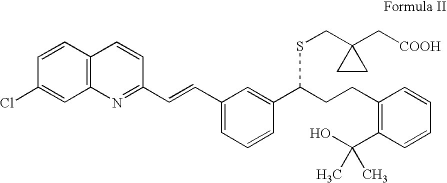
and stereoisomers, analogs, and pharmaceutical salts thereof.

10. The method of claim 7, wherein the leukotriene antagonists is selected from the following formula: 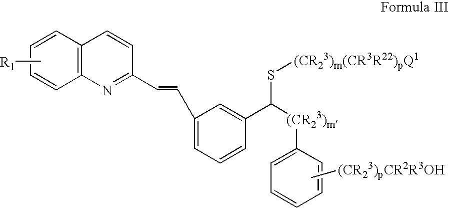
wherein:
R¹ is H, halogen, CF₃, or CN;
R²² is R³, —CH₂O₃, or —CH₂SR²;
Q¹ is —C(O)OH, 1H(or 2H)-tetrazol-5-yl, —C(O)NHS(O)₂R¹³, —C(O)NR¹²R¹², or —NHS(O)₂R¹³;
m′is 0, 1, 2 or 3;
p′ is 0 or 1;
m+p is 1-5;
the remaining definitions are as in Formula I;
and stereoisomers, analogs, and pharmaceutically acceptable salts thereof.

11. The method of claim 7, wherein the leukotriene antagonists is selected from the following formula: 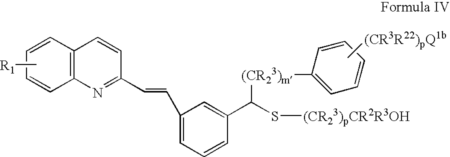
wherein:
R¹ is H, halogen, CF₃, or CN;
R²² is R³, —CH₂O₃, or —CH₂SR²;
Q¹ is —C(O)OH, 1H(or 2H)-tetrazol-5-yl, —C(O)NHS(O)₂R¹³, —C(O)NR¹²R¹², or —NHS(O)₂R¹³;
m′is 0, 1, 2 or 3;
p is 0 or 1
p′ 1-4;
m+p is 0-4;
the remaining definitions are as in Formula I;
and the pharmaceutically acceptable salts thereof.

12. The method of claim 7, wherein the leukotriene antagonist is selected from the group consisting of zafirlukast, montelukast, pranlukast, BAYx7195, LY293111, ICI 204,219, and ONO-1078.

13. A method of treating a patient who suffers from at least one of snoring or sleep apnea, comprising
periodic administration of at least one leukotriene antagonist, at a dosage and frequency which is effective in reducing at least one aspect of such snoring or sleep apnea in such patient.

14. The method of claim 13 wherein periodic administration of the leukotriene antagonist comprises periodic ingestion of an orally-ingestible unit dosage formulation of the leukotriene receptor-blocking drug.

15. The method of claim 13, wherein the leukotriene antagonist is selected from the group consisting of zafirlukast, montelukast, pranlukast, BAYx7195, LY293111, ICI 204,219, and ONO-1078.

16. The method of claim 13, wherein the leukotriene antagonists is selected from the following formula: 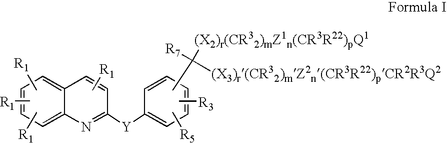
wherein:
R₁ is H, halogen, —CF₃, —CN, —NO₂, or N₃;
R₂ is lower alkyl, lower alkenyl, lower alkynyl, —CF₃, —CH₂F, —CH₂F₂, CH₂CF₃, substituted or unsubstituted phenyl, substituted or unsubstituted benzyl, substituted or unsubstituted 2-phenethyl, or two R² groups joined to the same carbon may form a ring of up to 8 members containing 0-2 heteroatoms chosen from O, S, and N;
R³ is H or R²;
CR³R²² may be the radical of a standard amino acid;
R⁴ is halogen, —NO₂, —CN, —OR₃, —SR³, NR³R³, NR³C(O)R⁷ or R³;
R⁵ is H, halogen, —NO₂, —N₃, —CN, —SR², —NR³R³, —OR³, lower alkyl, or —C(O)R³;
R⁶ is (CH₂)ₛ—C(R⁷R⁷)—CH₂)ₛ—R⁸ or —CH₂C(O)NR¹²R¹²;
R⁷ is H or C₁₋₄ alkyl;
R⁸ is
A) a monocyclic or bicyclic heterocyclic radical containing from 3 to 12 nuclear carbon atoms and 1 or 2 nuclear heteroatoms selected from N, S or O and with each ring in the heterocyclic radical being formed of 5 or 6 atoms, or
B) the radical W—R⁹;
R⁹ contains up to 20 carbon atoms and is (1) an alkyl group or (2) an alkylcarbonyl group of an organic acyclic or monocyclic carboxylic acid containing not more than 1 heteroatom in the ring;
R¹⁰ is —SR¹¹, —OR¹², or —NR¹²R¹²;
R¹¹ is lower alkyl, —C(O)R¹⁴, unsubstituted phenyl, or unsubstituted benzyl;
R¹² is H, R¹¹ or two R¹² groups joined to the same N may form a ring of 5 or 6 members containing 1-2 heteroatoms chosen from O, S, and N;
R¹³ is lower alkyl, lower alkenyl, lower alkynyl, —CF₃ or substituted or unsubstituted phenyl, benzyl, or 2-phenethyl;
R¹⁴ is H or R¹³;
R¹⁶ is H, C₁₋₄ alkyl, or OH;
R¹⁷ is lower alkyl, lower alkenyl, lower alkynyl, or substituted or unsubstituted phenyl, benzyl, or 2-phenethyl;
R¹⁸ is lower alkyl, lower alkenyl, lower alkynyl, —CF₃ or substituted or unsubstituted phenyl, benzyl, or 2-phenethyl;
R¹⁹ is lower alkyl, lower alkenyl, lower alkynyl, —CF₃ or substituted or unsubstituted phenyl, benzyl, or 2-phenethyl;
R²⁰ is H, C₁₋₄ alkyl, substituted or unsubstituted phenyl, benzyl, phenethyl, or pyridinyl or two
R²⁰ groups joined to the same N may form a saturated ring of 5 or 6 members containing 1-2 heteroatoms chosen from O, S, and N;
R²¹ is H or R¹⁷;
R²² is R⁴, CHR⁷OR³, or CHR⁷SR²;
m and m′ are independently 0-8;
n and m′ are independently 0 or 1;
p and p′ are independently 0-8;
m+n+p is 1-10 when r is 1 and X² is O, S, S(O), or S(O)₂;
m+n+p is 0-10 when r is 1 and X² is CR³R¹⁶;
m+n+p is 0-10 when r is O;
m′+m′+p′ is 0-10;
r and r′ are independently 0 or 1;
s is 0-3;
Q¹ is —C(O)OR³, 1H(or 2H)-tetrazol-5-yl, —C(O)OR⁶, —C(O)NHS(O)₂R¹³, —CN, —C(O)NR¹²R¹², —NR²¹(O)₂R¹², —CN, —NR¹²C(O)NR¹²R¹², —NR²¹C(O)R¹⁸—OC(O)NR¹²R¹², —C(O)R¹⁹, —S(O)R¹⁸, —S(O)₂R¹⁸, —S(O)₂NR¹²R¹², —NO₂, —NR²¹C(O)OR¹⁷, —C(NR¹²R¹²)═NR¹², —C(R¹³)═NOH; or if Q¹ —C(O)OH and R²² is —OH, —SH, —CHR⁷ OH or —NHR³, then Q¹ and R²² and the carbons through which they are attached may form a heterocyclic ring by loss of water;
Q² is OH or NR²⁰R²⁰;
W is O, S, or NR³;
X² and X³ are independently O, S, S(O), S(O)₂, or CR³R¹⁶;
Y is —CR³═CR³— or —C═C—;
Z¹ and Z² are independently —HET(—R³—R⁵)—;
HET is the diradical of a benzene, a pyridine, a furan, or a thiophene;
and stereoisomers, analogs, and pharmaceutically acceptable salts thereof.

17. The method of claim 13, wherein the leukotriene antagonists is selected from the following formula: 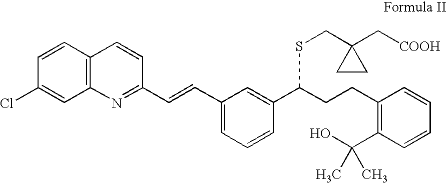
and stereoisomers, analogs, and pharmaceutical salts thereof.

18. The method of claim 13, wherein the leukotriene antagonists is selected from the following formula: 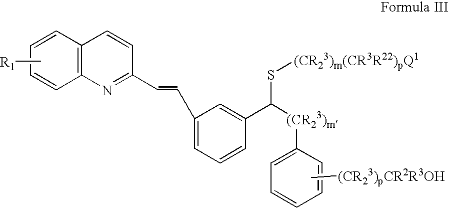
wherein:
R¹ is H, halogen, CF₃, or CN;
R²² is R³, —CH₂O₃, or —CH₂SR²;
Q¹ is —C(O)OH, 1H(or 2H)-tetrazol-5-yl, —C(O)NHS(O)₂R¹³, —C(O)NR¹²R¹², or —NHS(O)₂R¹³;
m′is 0, 1, 2 or 3;
p′ is 0 or 1;
m+p is 1-5;
the remaining definitions are as in Formula I;
and stereoisomers, analogs, and pharmaceutically acceptable salts thereof.

19. The method of claim 13, wherein the leukotriene antagonists is selected from the following formula: 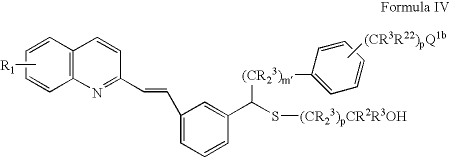
wherein:
R¹ is H, halogen, CF₃, or CN;
R²² is R³, —CH₂O₃, or —CH₂SR²;
Q¹ is —C(O)OH, 1H(or 2H)-tetrazol-5-yl, —C(O)NHS(O)₂R¹³, —C(O)NR¹²R¹², or —NHS(O)₂R¹³;
m′is Q, 1, 2 or 3;
p is 0 or 1
p′ is 1-4;
m+p is 0-4;
the remaining definitions are as in Formula I;
and the pharmaceutically acceptable salts thereof.

20. The method of claim 1, further comprising administration of a nasal steroid.

21. The method of claim 20, further comprising administration of a corticosteroid.

22. The method of claim 20, further comprising administration of a budesonide.

23. The method of claim 7, further comprising administration of a nasal steroid.

24. The method of claim 23, further comprising administration of a corticosteroid.

25. The method of claim 23, further comprising administration of a budesonide.

26. The method of claim 13, further comprising administration of a nasal steroid.

27. The method of claim 26, further comprising administration of a corticosteroid.

27. The method of claim 26, further comprising administration of a budesonide.

28. An article of manufacture comprising a leukotriene receptor-blocking drug inside a labeled package which encloses and protects the drug, wherein the leukotriene receptor-blocking drug is in an orally ingestible formulation which is effective in reducing snore and sleep apnea in it least some patients if taken chronically, and wherein the labeled package indicates to physicians and purchasers that the leukotriene receptor-blocking drug enclosed therein is effective, when administered periodically, in reducing at least one type of snoring and sleep apnea indications in at least some patients who suffer from such indications.

29. The article of manufacture of claim 28, wherein the orally ingestible formulation is a unit dosage form.

30. An article of manufacture, comprising:
a leukotriene antagonist, and
an intranasal corticosteroid.
